(19) United States
(12) Patent Application Publication
(10) Pub. No.: US 2023/0056797 A1
(43) Pub. Date: Feb. 23, 2023

(54) ENDOSCOPE SYSTEM, ENDOSCOPE, AND OPERATION UNIT

(71) Applicant: OLYMPUS MEDICAL SYSTEMS CORP., Tokyo (JP)

(72) Inventor: Yuta SATO, Tokyo (JP)

(73) Assignee: OLYMPUS MEDICAL SYSTEMS CORP., Tokyo (JP)

(21) Appl. No.: 17/408,378

(22) Filed: Aug. 21, 2021

(51) Int. Cl. A61B 1/005; A61B 1/00

(52) U.S. Cl. CPC A61B 1/0052; A61B 1/00016

(57) ABSTRACT

An endoscope system includes an endoscope including an insertion portion including a bending portion, a grasping portion arranged at a proximal end portion of the insertion portion, and an electric actuator configured to bend the bending portion, and an operation unit including an operation member configured to operate the electric actuator and a transmission member configured to wirelessly transmit operation information of the operation member to the electric actuator.

### BACKGROUND OF THE INVENTION

### 1. Field of the Invention

The present invention relates to an endoscope system including an endoscope including an electric actuator that bends a bending portion, an endoscope including an electric actuator that bends a bending portion, and an operation unit that bends a bending portion of an endoscope using an electric actuator.

### 2. Description of the Related Art

An endoscope includes a bending portion for changing an image pickup direction in an elongated insertion portion inserted into a subject. In a general endoscope, a surgeon turns an operation knob of a grasping portion, whereby a bending portion bends. In contrast, there has been proposed an endoscope that electrically performs bending operation.

Japanese Patent Application Laid-Open Publication No. H8 -224206 discloses an endoscope including a bending operation switch instead of an operation knob. The bending operation switch is selectively connected to, for example, a four connector receivers of a grasping portion. A user can select a position of the bending operation switch in the grasping portion according to preference by selecting a connector receiver in which the bending operation switch is arranged.

### SUMMARY OF THE INVENTION

An endoscope system in an embodiment includes: an endoscope including: an insertion portion including a bending portion; a grasping portion arranged on a proximal end side of the insertion portion; and an electric actuator configured to bend the bending portion; and an operation unit including: an operation member configured to operate the electric actuator; and a transmission member configured to wirelessly transmit operation information of the operation member to the electric actuator.

An endoscope in another embodiment includes: an insertion portion including a bending portion; a grasping portion arranged at a proximal end portion of the insertion portion; and an electric actuator configured to bend the bending portion based on operation information wirelessly transmitted from an operation unit.

An operation unit in another embodiment includes: an operation member configured to operate an electric actuator of an endoscope including: an insertion portion including a bending portion; a grasping portion arranged at a proximal end portion of the insertion portion; and the electric actuator configured to bend the bending portion; and a transmission member configured to wirelessly transmit operation information of the operation member to the electric actuator.

### BRIEF DESCRIPTION OF THE DRAWINGS

FIG. 1 is an exterior view of an endoscope system in an embodiment;

FIG. 2 is a sectional view of a distal end portion of an endoscope of the endoscope system in the embodiment;

FIG. 3 is a perspective exploded view of the distal end portion of the endoscope of the endoscope system in the embodiment;

FIG. 4 is a partial exterior view of the endoscope system in the embodiment;

FIG. 5 is a partial exterior view of the endoscope system in the embodiment;

FIG. 6 is a partial exterior view of an endoscope system in a modification 1;

FIG. 7A is a partial exterior view of an endoscope system in a modification 2;

FIG. 7B is a partial exterior view of the endoscope system in the modification 2;

FIG. 8 is a partial exterior view of an endoscope system in a modification 3;

FIG. 9 is an exterior view of an endoscope system in a modification 4;

FIG. 10 is a partial exterior view of an endoscope system in a modification 5; and

FIG. 11 is an exterior view of an endoscope system in a modification 6.

### DETAILED DESCRIPTION OF THE PREFERRED EMBODIMENT

### <Endoscope System>

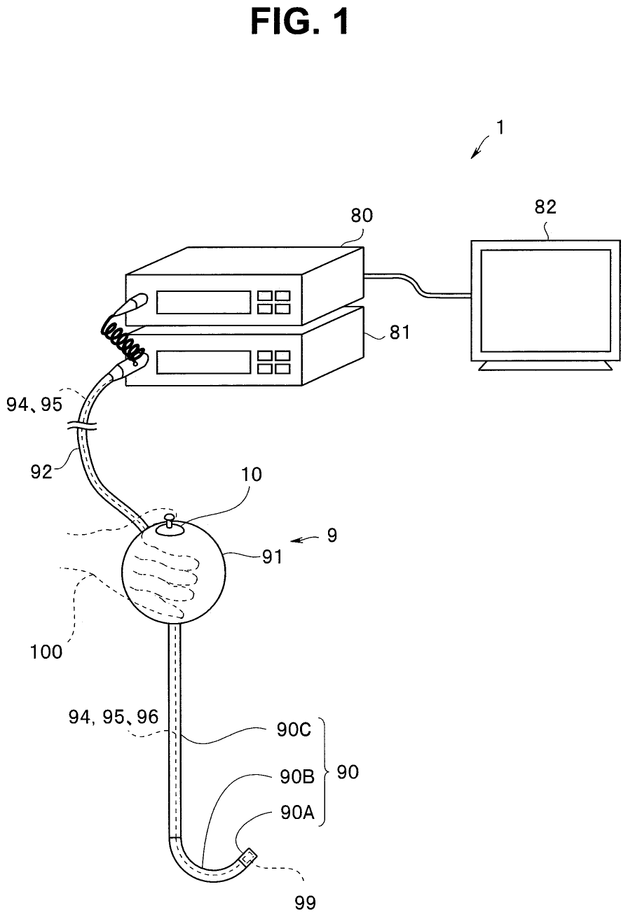

An endoscope system 1 in an embodiment shown in FIG. 1 includes an endoscope 9, an operation unit 10, an image processor 80, a light source apparatus 81, and a monitor 82. The endoscope 9 includes an insertion portion 90, a grasping portion 91, and a universal cord 92.

Note that drawings based on the embodiment are schematic diagrams. Relations between thicknesses and widths of respective portions, ratios of the thicknesses and relative angles of the respective portions, and the like are different from real ones. Portions, relations and ratios of dimensions of which are different, are included among the drawings. Illustration of a part of constituent elements and imparting of reference numerals and signs to a part of the constituent elements are omitted. A direction in which an object, an image of which is picked up, is present is referred to as “front, distal end side” and an opposite direction of “front, distal end side” is referred to as “rear, proximal end side”.

The insertion portion 90 of the endoscope 9 is configured by a distal end portion 90A where an image pickup unit 99 is arranged, a bending portion 90B consecutively connected to the distal end portion 90A and bending, and a straight pipe portion 90C consecutively connected to the bending portion 90B. In the endoscope 9, the insertion portion 90 is inserted into a body cavity of a subject and the image pickup unit 99 picks up an in-vivo image and outputs an image signal. The grasping portion 91 held by a hand 100 of a surgeon is arranged at a proximal end portion of the insertion portion 90 of the endoscope 9.

Since the grasping portion 91 is a sphere, the surgeon can easily hold the grasping portion 91 with the hand 100 in any posture. Cleaning treatment for the grasping portion 91 is easy. The grasping portion 91 may be an elliptical body. The grasping portion 91 may not be a perfect sphere or a perfect elliptical body and may be, for example, a substantial sphere or a substantial elliptical body, a part of which is formed by a plane. At least 70% or more of a surface of the grasping portion 91 only has to be formed by a curved surface.

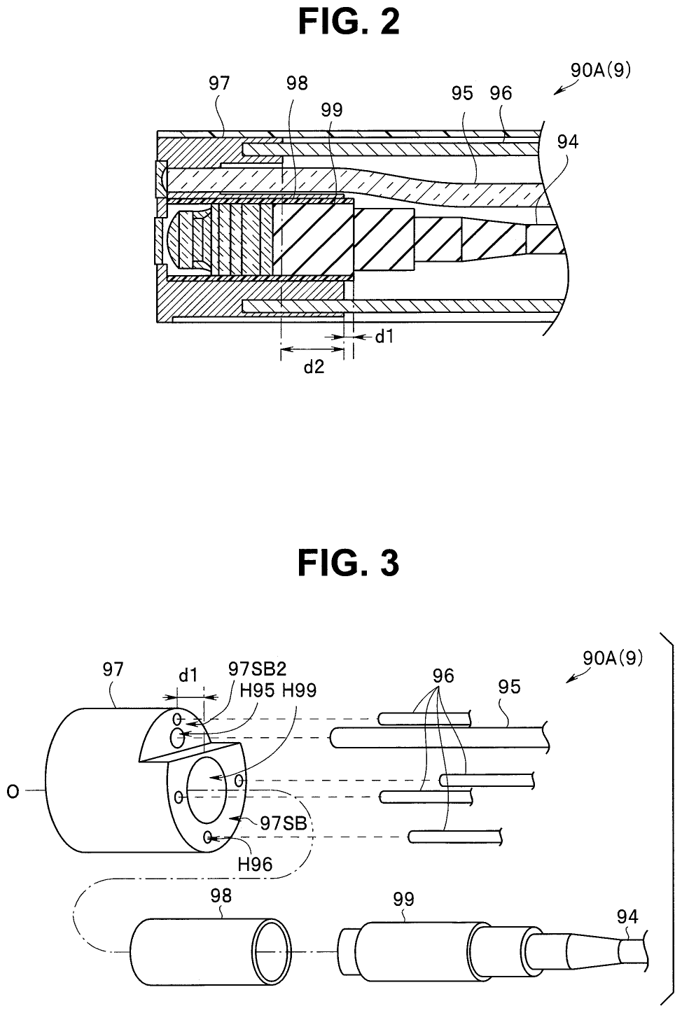

The light source apparatus 81 includes, for example, a white LED. Illumination light emitted by the light source apparatus 81 is guided to the distal end portion 90A by passing through a light guide 95 (see FIG. 2) inserted through the universal cord 92 and the insertion portion 90 to illuminate an object.

The universal cord 92, through which a signal cable 94 extended from the image pickup unit 99 is inserted, is connected to the image processor 80. The image processor 80 controls the entire endoscope system 1 and performs signal processing on an image pickup signal outputted by the image pickup unit 99 and outputs the image pickup signal as an image signal. The monitor 82 displays, as an endoscopic image, the image signal outputted by the image processor 80.

The endoscope 9 is a rigid endoscope for medical use. However, the endoscope of the present invention may be a flexible endoscope and use of the endoscope may be industrial use. The grasping portion 91 may have the same shape as a publicly known and used endoscope.

As shown in FIG. 2, a substantially columnar rigid member 97 is arranged at the distal end portion 90A. The image pickup unit 99 inserted into a cylinder 98 is inserted into a through-hole H99 of the rigid member 97 and is fixed using an adhesive (not shown). The image pickup unit 99 includes an image pickup device that photographs an object and outputs an image pickup signal. A rear end of the cylinder 98 made of an insulating body, into which the image pickup unit 99 is inserted, projects from a rear surface 97SB of the rigid member 97 (length d1). Accordingly, insulation of the image pickup unit 99 and the rigid member 97 is guaranteed by the cylinder 98.

The light guide 95 is inserted into a through-hole H95 of the rigid member 97. The rigid member 97 includes a cutout on the rear surface 97SB. In other words, a surface 97SB2 including an opening of the through-hole H95 is located farther on a distal end side by length d2 than the rear surface 97SB. Accordingly, the adhesive for fixing the image pickup unit 99 to the through-hole H99 does not intrude into the through-hole H95 into which the light guide 95 is inserted.

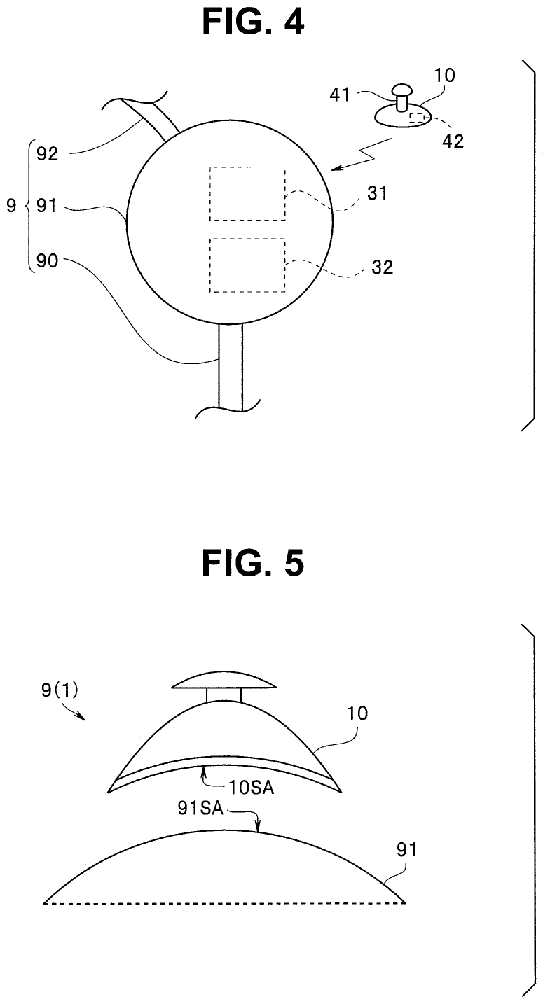

In the endoscope system 1, the bending portion 90B bends in four directions (upward, downward, left, and right) orthogonal to one another according to operation of the operation unit 10 by the surgeon. In other words, an electric actuator 31 that tows four bending wires 96 inserted into through-holes H96 of the rigid member 97 shown in FIG. 2 is arranged in the grasping portion 91 (FIG. 4).

The electric actuator 31 may be a motor or a heater, a Peltier element, or the like that heats bimetal arranged in the bending portion 90B. The bending portion 90B only has to bend in at least one direction.

The operation unit 10 includes a joystick 41, which is an operation member for operating the electric actuator 31, and a transmission circuit 42, which is a transmission member that wirelessly transmits operation information of the joystick 41 by the surgeon to the electric actuator 31 through a reception circuit 32.

The joystick 41 wirelessly transmits, using the transmission circuit 42, operation information corresponding to a direction in which the joystick 41 is tilted. The transmission circuit 42 is a short-range wireless communication circuit that performs transmission of data using a radio wave, for example, a WiFi circuit or Bluetooth (registered trademark).

The electric actuator 31 tows the bending wires 96 based on operation information received by the reception circuit 32, which is a reception member arranged in the grasping portion 91.

The surgeon can bend the bending portion 90B, which bends in the four directions orthogonal to one another, at a desired angle by operating the joystick 41 using one finger of the hand 100. The operation member may be a touch panel, a press switch, a potentiometer, or the like.

As shown in FIG. 5, one surface 10SA of the operation unit 10 is a suction cup having substantially the same curvature as a curvature of an outer surface 91SA of the grasping portion 91 and is stuck and fixed to the outer surface 91SA. Accordingly, the operation unit 10 can be fixed to any place of the grasping portion 91. The operation unit 10 including a magnet may be fixed to the grasping portion 91 made of a magnetic body.

It is likely that an operation unit fixed to a place selected out of several places of the grasping portion 91 cannot be fixed to an optimum place depending on a type of a procedure, a posture and a body shape (for example, size of the hand 100) of the surgeon, or the like.

In contrast, operation information of the joystick 41 of the operation unit 10 is wirelessly transmitted to the grasping portion 91. Therefore, an electric contact is not exposed to an outer surface of the grasping portion 91. Accordingly, it is unnecessary to consider deterioration of the electric contact by a medical solution or the like when the endoscope 9 is cleaned. Note that the operation unit 10 is detached from the endoscope 9 and cleaned separately from the endoscope 9.

The operation unit 10 can be fixed to any place of the grasping portion 91. Moreover, even when the posture of the surgeon changes after the fixing, the fixing position of the operation unit 10 can be adjusted. Accordingly, the endoscope system 1 has high operability.

As explained above, an endoscope in the embodiment of the present invention includes an insertion portion including a bending portion, a grasping portion arranged at a proximal end portion of the insertion portion, and an electric actuator configured to bend the bending portion based on operation information wirelessly transmitted from an operation unit.

An operation unit according to the embodiment of the present invention includes an operation member configured to operate an electric actuator of an endoscope including an insertion portion including a bending portion, a grasping portion arranged at a proximal end portion of the insertion portion, and the electric actuator configured to bend the bending portion, and a transmission member configured to wirelessly transmit operation information of the operation member to the electric actuator.

### <Modifications>

Endoscope systems 1A to 1F in modifications 1 to 6 are similar to the endoscope system 1 and have the same effects as the effects of the endoscope system 1. Accordingly, components having the same functions as the functions of the components in the endoscope system 1 are denoted by the same reference numerals and signs and explanation of the components is omitted.

### <Modification 1>

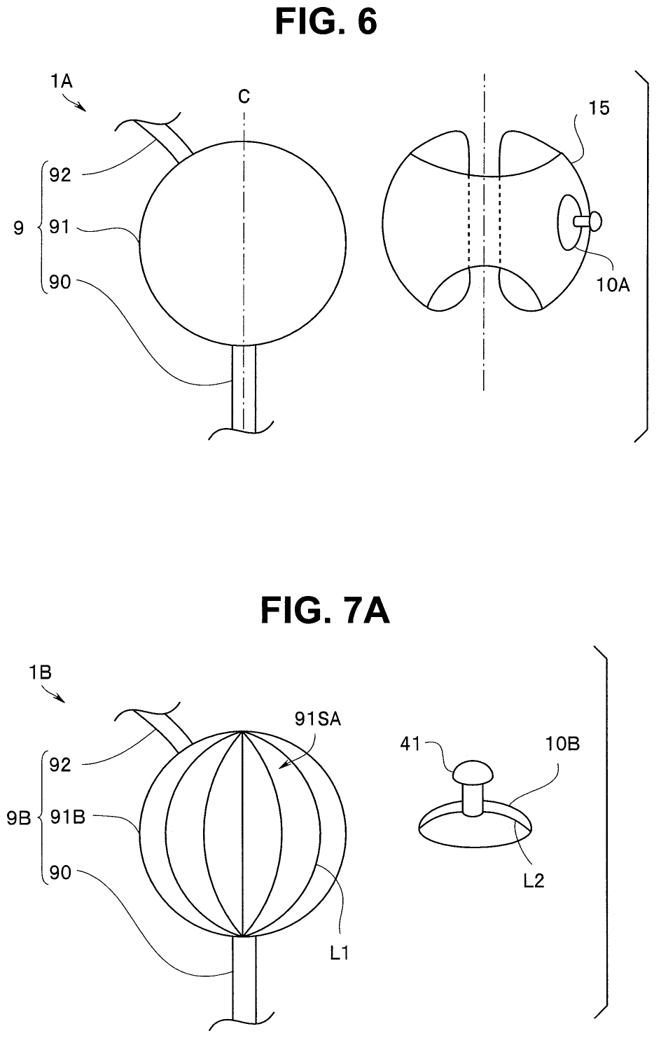

An operation unit 10A of the endoscope 9 of the endoscope system 1A shown in FIG. 6 is attached to a cover 15 arranged in the grasping portion 91. The cover 15 is made of, for example, an elastic material of silicone resin. The cover 15 includes an opening in a region where the insertion portion 90 and the universal cord 92 are connected. However, a relative position to the grasping portion 91 can be optionally arranged. In particular, the cover 15 can be arranged in any rotation position centering on an axis C virtually extended from a center axis of the insertion portion 90.

The cover 15 is easily detachably attachable to the grasping portion 91. The operation unit 10A may be arranged in an arrangement position of the operation unit 10A or in the cover 15 selected by the surgeon out of a plurality of covers 15, materials or the like of which are different.

The operation unit 10A is arranged in the cover 15 easily fixed to the insertion portion 90. Therefore, the operation unit 10A is easily attached to and detached from the grasping portion 91.

### <Modification 2>

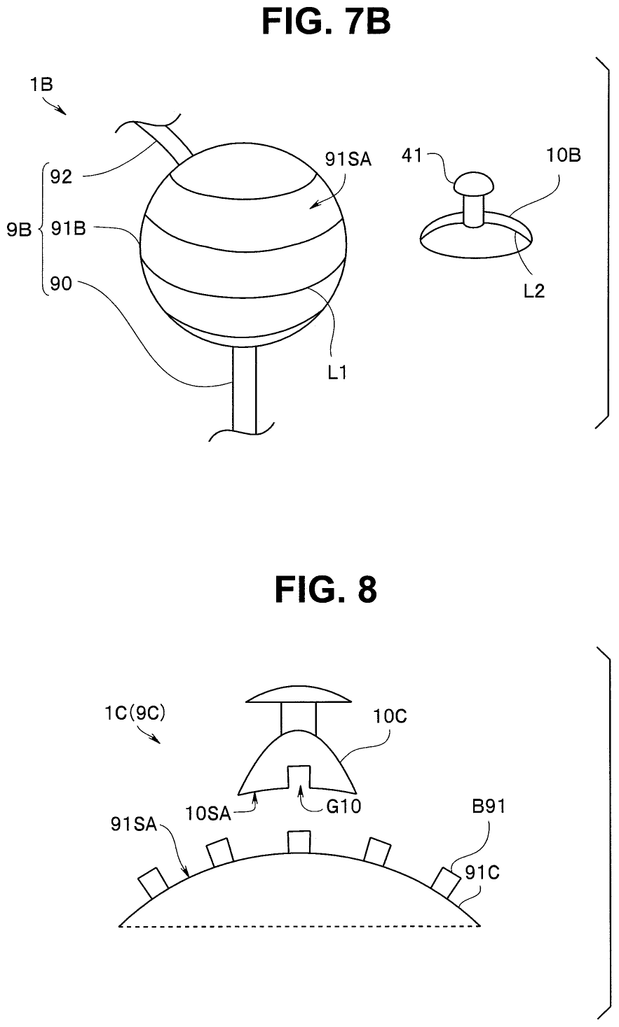

In the endoscope system 1B in this modification shown in FIG. 7A, a linear first mark L1 is attached to the outer surface 91SA of a grasping portion 91B of an endoscope 9B. Meanwhile, a linear second mark L2 is attached to an operation unit 10B.

A direction of a line of the first mark L1 corresponds to up-down bending of the bending portion 90B. A direction of a line of the second mark L2 corresponds to a bending direction of the bending portion 90B corresponding to an operation direction of the joystick 41.

In the endoscope system 1B in this modification shown in FIG. 7B, a direction of a line of the first mark L1 on the outer surface 91SA of the grasping portion 91 of the endoscope 9B corresponds to left-right bending of the bending portion 90B.

In the endoscope system 1B, the operation unit 10B is arranged in a state in which the second mark L2 is parallel to the first mark L1 or a state in which the second mark L2 is parallel to a part of the first mark L1, whereby an operation direction of the operation unit 10B and a bending direction of the bending portion 90B can be matched. Accordingly, the endoscope system 1B is easily attached to the grasping portion in a state in which the operation direction of the operation unit coincides with the bending direction of the bending portion 90B.

Note that it goes without saying that marks respectively corresponding to the up-down bending and the left-right bending of the bending portion 90B may be attached to the outer surface 91SA of the grasping portion 91B.

### <Modification 3>

In the endoscope system 1C in this modification shown in FIG. 8, elongated protrusions B91 corresponding to the bending direction of the bending portion 90B are present on the outer surface 91SA of a grasping portion 91C. Meanwhile, a groove (an elongated recess) G10 is present on one surface of an operation unit 10C. An extending direction of the elongated protrusions B91 corresponds to the bending direction (an up-down direction or a left-right direction) of the bending portion 90B. A major axis direction of the groove G10 corresponds to the bending direction of the bending portion 90B corresponding to the operation direction of the joystick 41.

The endoscope system 1C has the same effects as the effects of the endoscope system 1B. Further, since the protrusions B91 and the groove G10 fit each other, the operation unit 10C is fixed. Accordingly, the endoscope system 1C is easily attached to the grasping portion 91C of the operation unit 10C.

Note that a groove may be present on the outer surface 91SA of the grasping portion 91C and a protrusion fitting into the groove may be present on one surface of the operation unit 10C.

### <Modification 4>

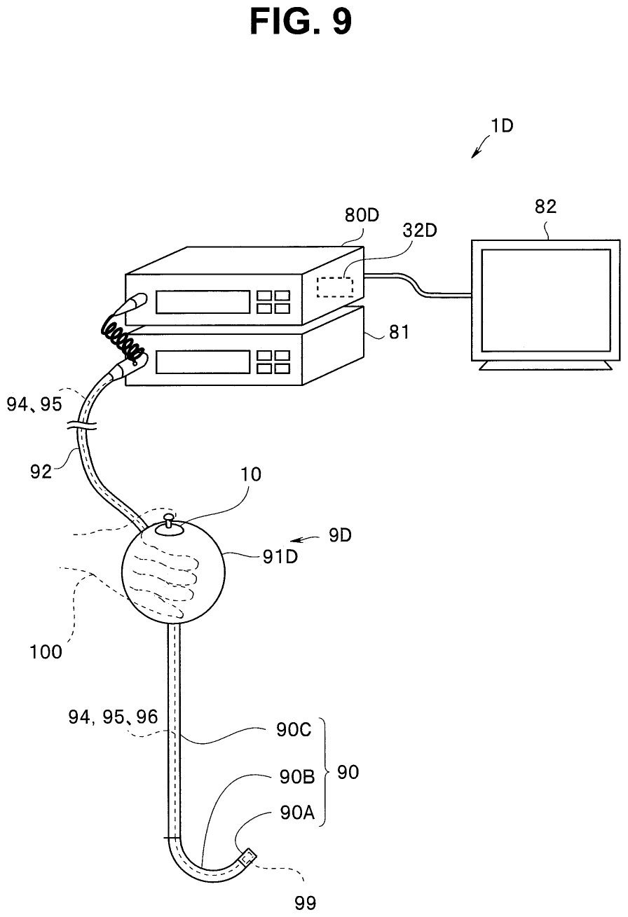

In the endoscope system 1D in this modification shown in FIG. 9, an image processor 80D includes a reception circuit 32D that receives a wireless signal, which is operation information of the operation unit 10. The operation information of the operation unit 10 is transmitted to the electric actuator 31 in a grasping portion 91D through the universal cord 92.

When an endoscope 9D is subjected to autoclave treatment, the grasping portion 91D also has high temperature. However, in the endoscope system 1D, since the reception circuit 32D does not have high temperature, reliability is high.

### <Modification 5>

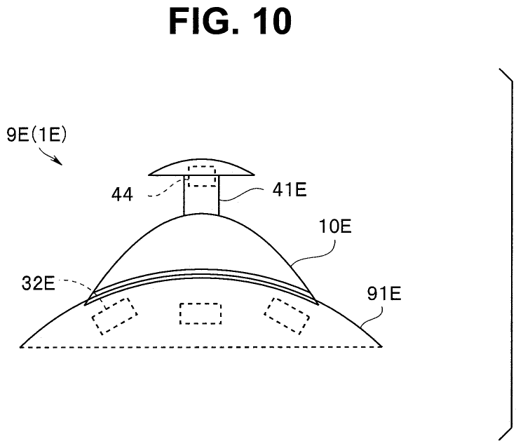

In the endoscope system 1E in this modification shown in FIG. 10, a joystick 41E, which is an operation unit for operating the electric actuator 31 of an operation unit 10E, includes a magnet 44. A magnetic field sensor 32E that detects a magnetic field of the magnet 44 is arranged in a grasping portion 91E.

For example, a magnetic field from the magnet 44 is detected by four Hall elements 32E arranged in rotationally symmetrical positions, whereby a position change of the magnet 44 based on operation of the joystick 41E is detected.

Operation information of the joystick 41E is wirelessly transmitted to the grasping portion 91E. Accordingly, the endoscope system 1E has the same effects as the effects of the endoscope system 1. Further, power supply to the operation unit 10E is unnecessary.

Note that, instead of the operation unit 10E, a touch sensor for operating the electric actuator may be arranged on a surface of the grasping portion 91E in a state in which an electrode is not exposed to an outer surface.

### <Modification 6>

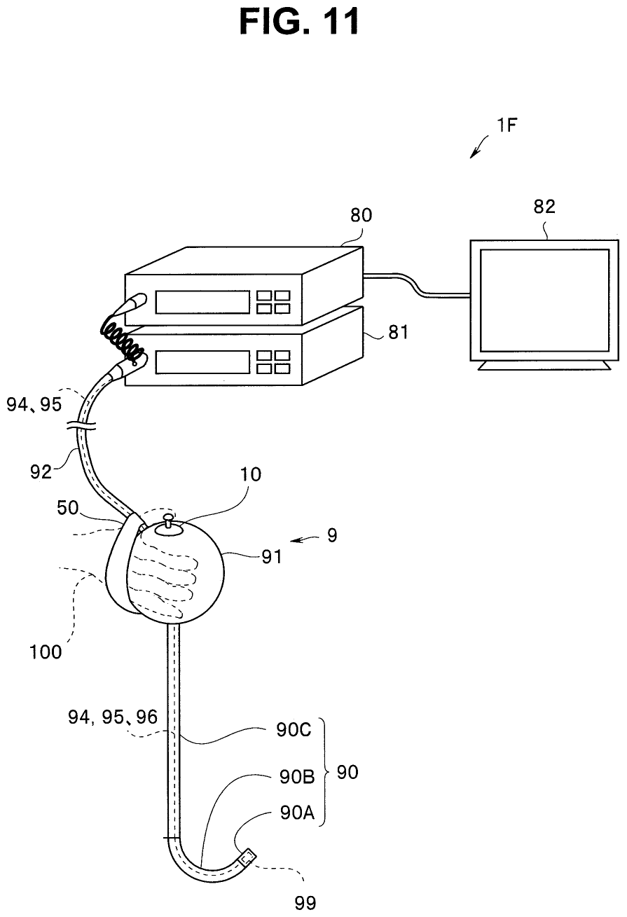

The endoscope system 1F in this modification shown in FIG. 11 includes a belt body 50 for fixing the hand 100 of the surgeon to the grasping portion 91.

The belt body 50 is, for example, a rubber band having elasticity or a cloth including a surface fastener. In the endoscope system 1F, the surgeon does not need to grip and fix the grasping portion 91. Accordingly, a movable range of the hand 100 for operating the operation unit 10 is expanded and operability is improved. Fatigue of the surgeon is reduced by the belt body 50.

The belt body 50 is removably arranged in the universal cord 92 but may be arranged in the grasping portion 91 or the like. The belt body 50 is detached from the endoscope 9 when the endoscope 9 is cleaned.

The present invention is not limited to the embodiment and the like explained above. Various changes, combinations, and applications are possible within a range not departing from the gist of the invention.

What is claimed is:

1. An endoscope system comprising:
an endoscope comprising:
an insertion portion including a bending portion;
a grasping portion arranged on a proximal end side of the insertion portion; and
an electric actuator configured to bend the bending portion; and
an operation unit comprising:
an operation member configured to operate the electric actuator; and
a transmission member configured to wirelessly transmit operation information of the operation member to the electric actuator.

2. The endoscope system according to claim 1, wherein
the transmission member is a transmission circuit configured to transmit the operation information using a wireless signal, and
the endoscope comprises a reception circuit configured to receive the wireless signal.

3. The endoscope system according to claim 1, further comprising an image processor electrically connected with the grasping portion of the endoscope via a universal cord, wherein
the transmission member is a transmission circuit configured to transmit the operation information using a wireless signal,
the image processor comprises a reception circuit configured to receive the wireless signal, and
the operation information is transmitted to the electric actuator via the universal cord.

4. The endoscope system according to claim 1, wherein
a magnet that moves according to operation of the operation member of the operation unit is the transmission member, and
the endoscope comprises a magnetic sensor configured to detect a magnetic field change of the magnet, which is the operation information.

5. The endoscope system according to claim 1, wherein the operation unit is fixed in any position on a surface of the grasping portion.

6. The endoscope system according to claim 1, wherein the endoscope system comprises a belt body for fixing a hand of a surgeon to the grasping portion.

7. The endoscope system according to claim 1, wherein the grasping portion is a substantial sphere or a substantial elliptical body.

8. The endoscope system according to claim 1, wherein
a first mark corresponding to a bending direction of the bending portion is attached to the grasping portion, and
a second mark corresponding to an operation direction is attached to the operation unit.

9. The endoscope system according to claim 1, wherein
an elongated recess or an elongated projection corresponding to a bending direction of the bending portion is present in the grasping portion, and
an elongated projection or an elongated recess that corresponds to an operation direction and fits into the recess or the projection is present in the operation unit.

10. An endoscope comprising:
an insertion portion including a bending portion;
a grasping portion arranged at a proximal end portion of the insertion portion; and
an electric actuator configured to bend the bending portion based on operation information wirelessly transmitted from an operation unit.

11. An operation unit comprising:
an operation member configured to operate an electric actuator of an endoscope including an insertion portion including a bending portion, a grasping portion arranged at a proximal end portion of the insertion portion, and the electric actuator configured to bend the bending portion; and
a transmission member configured to wirelessly transmit operation information of the operation member to the electric actuator.
